(19) United States
(12) Patent Application Publication
(10) Pub. No.: US 2005/0250779 A1
(43) Pub. Date: Nov. 10, 2005

(54) Pteridinone derivatives for treating ocular hypertension

(76) Inventor: Song Zhu, Bedminster, NJ (US)

Correspondence Address: SL LABORATORIES, LLC, 1430 ROUTE 206, SUITE 210, BEDMINSTER, NJ, 07921, US

(21) Appl. No.: 10/838,479

(22) Filed: May 4, 2004

(51) Int. Cl. C07D 475/02; A61K 31/525

(52) U.S. Cl. 514/251; 544/257

(57) ABSTRACT

This invention relates to potent potassium channel blocker compounds of Formula I or a formulation thereof for the treatment of glaucoma and other conditions which lead to elevated intraoccular pressure in the eye of a patient. This invention also relates to the use of such compounds to provide a neuroprotective effect to the eye of mammalian species, particularly humans.

### BACKGROUND OF THE INVENTION

Glaucoma is a common condition in which there is a build-up of intraocular pressure (IOP) in the eye. This may cause eye and head pain, haloes in the vision, constriction of the visual field, or merely a progressive loss of vision without other symptoms. It is the major cause of irreversible blindness in Western societies. A variety of oral medicines or eye drops are customarily employed, but are not uniformly effective.

Maxi-K channels are large conductance, voltage and calcium-sensitive potassium channels which are fundamental to the control of smooth muscle tone and neuronal excitability. Maxi-K channels can be formed by 2 subunits: the pore-forming alpha subunit and the modulatory beta subunit.

The calcium and voltage gated, high conductance potassium channel (maxi-K) plays a central role in restoring the resting potential of excitable cells. This action in smooth muscle cells is important in setting vascular tone; consequently pharmacological manipulation of maxi-K channels remains a potential route for management of hypertension.

There are several current therapies for treatment of glaucoma and elevated intraocular pressure, but the efficacy and the side effect profiles of these agents are unsatisfactory. Recently potassium channel blockers were found reducing intraocular pressure in the eye and therefore provide one more approach to the treatment of ocular hypertension and the degenerative ocular conditions related thereto. Blockage of potassium channels can provide a neuroprotective effect to the eye of mammalian species, particularly humans. Therefore, Maxi-K channel blockers can be used for the treatment of glaucoma and/or ocular hypertension (elevated intraocular pressure). (see U.S. Pat. Nos. 5,573,758 and 5,925,342; Moore, et al., Invest. Ophthalmol. Vis Sci 38, 1997; WO 89/10757, WO94/28900, and WO 96/33719).

### SUMMARY OF THE INVENTION

This invention relates to the use of potent potassium channel blockers or a formulation thereof in the treatment of glaucoma and other conditions which are related to elevated intraocular pressure in the eye of a patient. This invention also relates to the use of such compounds to provide a neuroprotective effect to the eye of mammalian species, particularly humans. More particularly this invention relates to the treatment of glaucoma and/or ocular hypertension (elevated intraocular pressure) using novel pteridinone compounds having the structural formula I: 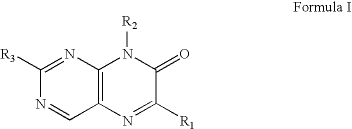
or a pharmaceutically acceptable salt, enantiomer, diastereomer or mixture thereof: wherein,
R₁ represents hydrogen, C₁₋₁₀ alkyl, —C(O)Ra, —(CHRₐ)ₙCONRbRc, —(CH₂)ₙRa, —(CH₂)ₙC₃₋₁₀ heterocyclyl, —(CH₂)ₙC₃₋₈ cycloalkyl, —COORa, aryl, heterocyclyl and alkyl optionally substituted with 1-3 groups selected from R^{a};
R₂ represents hydrogen, C₁₋₁₀ alkyl, C₂₋₆ alkenyl, C₁₋₆ alkylSRa, —(CH₂)ₙO(CH₂)ₘOR, —(CH₂)ₙC₁₋₆ alkoxy, —(CH₂)ₙC₃₋₈ cycloalkyl, —(CH₂)ₙC₃₋₁₀ heterocyclyl, or —(CH₂)ₙRa, said alkyl, heterocyclyl, or aryl optionally substituted with 1-3 groups selected from R^{b};
R₃ represents hydrogen, C₁₋₁₀ alkyl, —(CH₂)ₙC₃₋₈ cycloalkyl, —(CH₂)ₙC₃₋₁₀ heterocyclyl, —(CH₂)ₙNHRₐ, —(CH₂)ₙN(Ra)₂, aryl, C₁₋₆ alkoxy, CF₃, —OCH3, (CH₂)ₙSO₂Ra, —(CH₂)ₙSO₂N(Ra)₂, —(CH₂)ₙCON(Ra)₂, —(CH₂)ₙCONHC(Ra)₃, nitro, cyano or halogen, said allyl, alkoxy, heterocyclyl, or aryl optionally substituted with 1-3 groups of R^{a};
Ra represents hydrogen, or C₁₋₁₀ alkyl, C4-12 aryl, C₁₋₁₀ alkyl, —(CH₂)ₙC₃₋₈ cycloalkyl, —(CH₂)ₙC₃₋₁₀ heterocyclyl.
Rb and Rc independently represent H, Ra, C₂₋₆ alkenyl, C₁₋₆ alkylSRa, —(CH₂)ₙO(CH₂)ₘORa, —(CH₂)ₙC₁₋₆ alkoxy, —(CH₂)ₙC₃₋₈ cycloalkyl;
n=1-6

### DETAILED DESCRIPTION OF THE INVENTION

The compounds which are utilized in accordance with the present invention, and in pharmaceutical compositions of the present invention, are novel potassium channel blockers of Formula I. It is also relates to a method for lowering elevated intraocular pressure (IOP) or treating glaucoma by administration in a pharceutically acceptable carrier in sufficiet concentration so as to deliver an effective amount of the active compound or compounds in the invention to the eye, derectlt or indirectly, preferably topical or intra-cammaral administration Preferably the ophthalmic, therapeutic solutions contains one or more of the compounds in the invention in a concentration range of approximatelly 0.00001% to approximately 1% (weight by volume), more preferably approximately 0.0005% to approximately 0.1% (weight by volume).

The present invention is directed to novel compounds of Formula (I), or a pharmaceutically acceptable salt, enantiomer, diastereomer or mixture thereof: 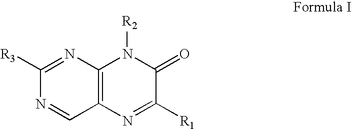
or a pharmaceutically acceptable salt, enantiomer, diastereomer or mixture thereof: wherein,
R₁ represents hydrogen, C₁₋₁₀ alkyl, —C(O)Ra, —(CHRₐ)ₙCONRbRc, —(CH₂)ₙRa, —(CH₂)ₙC₃₋₁₀ heterocyclyl, —(CH₂)ₙC₃₋₈ cycloalkyl, —COORa, aryl, heterocyclyl and alkyl optionally substituted with 1-3 groups selected from R^{a};
R₂ represents hydrogen, C₁₋₁₀ alkyl, C₂₋₆ alkenyl, C₁₋₆ alkylSRa, —(CH₂)ₙO(CH₂)ₘOR, —(CH₂)ₙC₁₋₆ alkoxy, —(CH₂)ₙC₃₋₈ cycloalkyl, —(CH₂)ₙC₃₋₁₀ heterocyclyl, or —(CH₂)ₙRa, said alkyl, heterocyclyl, or aryl optionally substituted with 1-3 groups selected from R^{b};
R₃ represents hydrogen, C₁₋₁₀ alkyl, —(CH₂)ₙC₃₋₈ cycloalkyl, —(CH₂)ₙC₃₋₁₀ heterocyclyl, —(CH₂)ₙNHRₐ, —(CH₂)ₙN(Ra)₂, aryl, C₁₋₆ alkoxy, CF₃, —OCH3, —(CH₂)ₙSO₂Ra, —(CH₂)ₙSO₂N(Ra)₂, —(CH₂)ₙCON(Ra)₂, —(CH₂)ₙCONHC(Ra)₃, nitro, cyano or halogen, said alkyl, alkoxy, heterocyclyl, or aryl optionally substituted with 1-3 groups of R^{a};
Ra represents hydrogen, or C₁₋₁₀ alkyl, C4-12 aryl, C₁₋₁₀ alkyl, —(CH₂)ₙC₃₋₈ cycloalkyl, —(CH₂)ₙC₃₋₁₀ heterocyclyl.
Rb and Rc independently represent H, Ra, C₂₋₆ alkenyl, C₁₋₆ alkylSRa, —(CH₂)ₙO(CH₂)ₘORa, —(CH₂)ₙC₁₋₆ alkoxy, —(CH₂)ₙC₃₋₈ cycloalkyl; n−1-6

Examples of compounds to be used in this invention are found in Table 1:

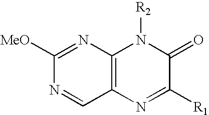

| R2 | R1 |
|---|---|
| Methyl | t-butyl |
| Methyl | Isopropyl |
| Ethyl | t-butyl |
| Ethyl | Isopropyl |
| Propyl | t-butyl |
| Propyl | Isopropyl |
| n-Butyl | Isopropyl |
| n-Butyl | t-butyl |
| Isopropyl | Isopropyl |
| Isopropyl | t-butyl |
| Benzyl | t-butyl |
| Benzyl | Isopropyl |
| 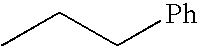 | t-butyl |
| 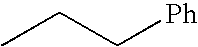 | Isopropyl |
| 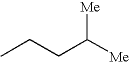 | t-butyl |
| 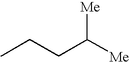 | Isopropyl |
| 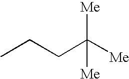 | t-butyl |
| 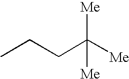 | Isopropyl |

The compounds of the present invention may have asymmetric centers, chiral axes and chiral planes, and occur as racemates, racemic mixtures, and as individual diastereomers, with all possible isomers, including optical isomers, being included in the present invention.

The invention is described herein in detail using the terms defined below unless otherwise specified.

When any variable (e.g. aryl, heterocycle, R¹, R⁶ etc.) occurs more than one time in any constituent, its definition on each occurrence is independent at every other occurrence. Also, combinations of substituents/or variables are permissible only if such combinations result in stable compounds.

The term “Halogen (halo)” is used herein to mean the chloro, fluoro, bromo, or iodo.

“Cycloalkyl” is used herein to mean cyclic radicals, preferably 3-8 carbons. It may contain from 1 to 4 rings, which are fused. Examples of such cycloalkyl elements include, but are not limited to, cyclopropyl, cyclobutyl, cyclopentyl, cyclohexyl and the like.

The term “cycloalkenyl” is used herein to mean cyclic radicals, preferably 5 tp 10 carbons, which at least one bond including but not limited to cyclopentenyl, cyclohexenyl, and thee like.

The term “heterocyclyl” or “heterocyclic”, on it's own or in any combination, such as “heteroaryloxy”, or “heroaryl alkyl”, as used herein, represents a stable 3- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. A fused heterocyclic ring system may include carbocyclic rings and need include only one heterocyclic ring. The term heterocycle or heterocyclic includes heteroaryl moieties. Examples of such heterocyclic elements include, but are not limited to, 2-azepinonyl, benzimidazolyl, benzisoxazolyl, benzofurazanyl, benzopyranyl, benzothiopyranyl, benzofuryl, benzothiazolyl, benzothienyl, benzoxazolyl, chromanyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, piperidyl, piperazinyl, pyridyl, pyrazinyl, pyrazolyl, pyridazinyl, pyrimidinyl, pyrrolidinyl, pyrrolyl, quinazolinyl, quinolinyl, tetrahydrofuryl, tetrahydroquinolinyl, thiamorpholinyl, thiazolyl thiazolinyl, and thienyl. Preferably, heterocycle is selected from 2-azepinonyl, benzimidazolyl, 2-diazapinonyl, dihydroimidazolyl, dihydropyrrolyl, imidazolyl, 2-imidazolidinonyl, indolyl, isoquinolinyl, morpholinyl, piperidyl, piperazinyl, pyridyl, pyrrolidinyl, 2-piperidinonyl, 2-pyrimidinonyl, 2-pyrollidinonyl, quinolinyl, tetrahydrofuryl, tetrahydroisoquinolinyl, and thienyl.

The term “heteroatom” means O, S or N, selected on an independent basis.

The term “heteroaryl” (on its own or in any combination, such as “heteroaryloxy”, or “heteroaryl alkyl”) is used herein to mean a 5-10 membered aromatic ring system in which one or more rings contain one or more herteroatoms selected from the group consisting of N, O or S, such as, but not limited, to pyrrole, pyrazole, furan, pyran, thiophene. quinoline, isoquinoline, quinazolinyl, pyridine, pyrimidine, pyridazine, pyrazine, uracil oxadiazole, oxazole, isoxazole, oxathiadiazole, thiazole, isothiazole, thiadiazole, tetrazole, triazole, indazole, imidazole, or benzimidazole.

The term “alkyl” refers to a monovalent alkane (hydrocarbon) derived radical containing from 1 to 10 carbon atoms unless otherwise defined. It may be straight, branched or cyclic. Preferred alkyl groups include methyl, ethyl, propyl, isopropyl, butyl, t-butyl, cyclopropyl cyclopentyl and cyclohexyl. When the alkyl group is said to be substituted with an alkyl group, this is used interchangeably with “branched alkyl group”.

“Alkenyl” is C₂-C₆ alkenyl.

“Alkoxy” refers to an alkyl group of indicated number of carbon atoms attached through an oxygen bridge, with the alkyl group optionally substituted as described herein. Said groups are those groups of the designated length in either a straight or branched configuration and if two or more carbon atoms in length, they may include a double or a triple bond. Exemplary of such alkoxy groups are methoxy, ethoxy, propoxy, isopropoxy, butoxy, isobutoxy, tertiary butoxy, pentoxy, isopentoxy, hexoxy, isohexoxy allyloxy, propargyloxy, and the like.

“Aryl” refers to aromatic rings e.g., phenyl, substituted phenyl and the like, as well as rings which are fused, e.g., naphthyl, phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. Examples of aryl groups are phenyl, naphthyl, tetrahydronaphthyl, indanyl, biphenyl, phenanthryl, anthryl or acenaphthyl and phenanthrenyl, preferably phenyl, naphthyl or phenanthrenyl. Aryl groups may likewise be substituted as defined Preferred substituted aryls include phenyl and naphthyl.

This invention is also concerned with a method of treating ocular hypertension or glaucoma by administering to a patient in need thereof one of the compounds of formula I, on its own or in combination with other pharceutically acceptable medications.

Intraocular pressure (IOP) is controlled by aqueous humor dynamics. Aqueous humor is produced at the level of the non-pigmented ciliary epithelium and is cleared primarily via outflow through the trabecular meshwork. Aqueous humor inflow is controlled by ion transport processes. It is thought that maxi-K channels in non-pigmented ciliary epithelial cells indirectly control chloride secretion by two mechanisms; these channels maintain a hyperpolarized membrane potential (interior negative) which provides a driving force for chloride efflux from the cell, and they also provide a counter ion (K⁺) for chloride ion movement. Water moves passively with KCl allowing production of aqueous humor. Inhibition of maxi-K channels in this tissue would diminish inflow. Maxi-K channels have also been shown to control the contractility of certain smooth muscle tissues, and, in some cases, channel blockers can contract quiescent muscle, or increase the myogenic activity of spontaneously active tissue. Contraction of ciliary muscle would open the trabecular meshwork and stimulate aqueous humor outflow, as occurs with pilocarpine. Therefore maxi-K channels could profoundly influence aqueous humor dynamics in several ways; blocking this channel would decrease IOP by affecting inflow or outflow processes or by a combination of affecting both inflow/outflow processes.

The calcium and voltage gated, high conductance potassium channel (maxi-K) plays a central role in restoring the resting potential of excitable cells. This action in smooth muscle cells is important in setting vascular tone; consequently pharmacological manipulation of maxi-K channels remains a potential route for management of hypertension.

The present invention is based upon the finding that maxi-K channels, if blocked, inhibit aqueous humor production by inhibiting net solute and H₂O efflux and therefore lower IOP. This finding suggests that maxi-K channel blockers are useful for treating other ophthamological dysfunctions such as macular edema and macular degeneration. It is known that lowering IOP promotes blood flow to the retina and optic nerve. Accordingly, the compounds of this invention are useful for treating macular edema and/or macular degeneration.

Glaucoma is a common condition in which there is a build-up of intraocular pressure (IOP) in the eye. This may cause eye and head pain, haloes in the vision, constriction of the visual field, or merely a progressive loss of vision without other symptoms. It is the major cause of irreversible blindness in Western societies. It is believed that maxi-K channel blockers which lower IOP are useful for providing a neuroprotective effect. They are also believed to be effective for increasing retinal and optic nerve head blood velocity and increasing retinal and optic nerve oxygen by lowering IOP, which when coupled together benefits optic nerve health. As a result, this invention further relates to a method for increasing retinal and optic nerve head blood velocity, increasing retinal and optic nerve oxygen tension as well as providing a neuroprotective effect or a combination thereof. Each of the claimed compounds are potassium channel antagonists and are thus useful in the neurological disorders in which it is desirable to maintain the cell in a depolarized state to achieve maximal neurotransmitter release. The compounds produced in the present invention are readily combined with suitable and known pharmaceutically acceptable excipients to produce compositions which may be administered to mammals, including humans, to achieve effective potassium channel blockage.

For use in medicine, the salts of the compounds of formula I will be pharmaceutically acceptable salts. Other salts may, however, be useful in the preparation of the compounds according to the invention or of their pharmaceutically acceptable salts. When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, isopropylamine, lysine, morpholine, piperazine, piperidine, polyamine resins, procaine, purines, theobromine, triethylamine, trimethylamine tripropylamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The ophthalmic solution or suspension may be administered as often as necessary to maintain an acceptable IOP level in the eye. It is contemplated that administration to the mamalian eye will be about once or twice daily.

The following examples given by way of illustration is demonstrative of the present invention.

Definitions of the terms used in the examples are as follows:
SM—starting material,
DMSO—dimethyl sulfoxide,
TFA—trifluoroacetic acid,
TLC—thin layer chromatography,
PTLC—preparative thin layer chromatography
h=hr=hour,
THF—tetrahydrofuran,
DMF—dimethylformamide,
min—minute,
LC/MS—liquid chromatography/mass spectrometry,
HPLC—high performance liquid chromatography,
EDC—1-(3-dimethylaminopropyl)-3-ethylcarbodiimide hydrochloride
HOBT—1-hydroxybenzotriazole
equiv=eq=equivalent,
rt=r.t=RT—room temperature,
psi—pounds per square inch

The compounds of this invention generally can be made, with modification where appropriate, in accordance with Schemes. Examples 1-24 are also produced in accordance with Schemes 1.

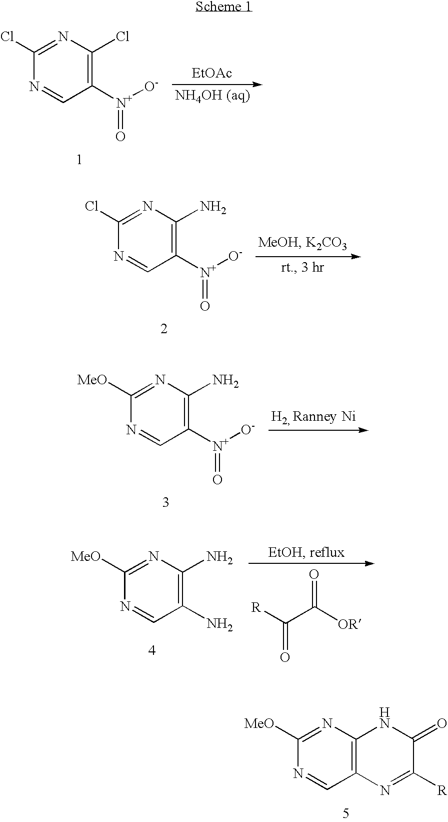

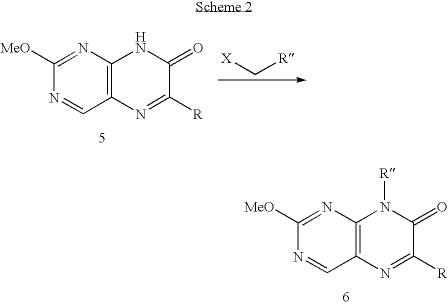

In Scheme 1, treatment of 2,4-dichloro-5-nitro-pyrimidine 1 with amonia hydroxide gave 2. Another chlorine was replaced by stirring 2 in methanol containing potassium carbonate. Reduction of 3 by hydrogenation gave diamine 4. Refluxing 4 in ethanol with appropriate ketoacid or ketoester produce pteridine 5.

In Scheme 2, compound 5 reacted with halide, such as alkyl bromide or alkyl iodide, can generated a series of analogs by replacement (alkylation) reaction, which are maxi-K inhibitors.

### PREPARATIVE EXAMPLE 1

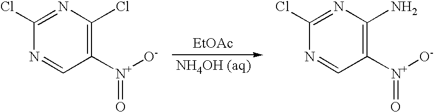

To a solution of 5 g (25 mmol) 2,4-dichloro-5-nitro-pyrimdine in 150 mL EtOAc was added 50 mL 28-34% aqueous NH₄OH. After stirred at rt for 1 hour, the reaction mixture was filtered. The organic layer was concentrated to give desired solid product. 1H NMR (CD₃OD): 9.03 (1H, s). LC-MS [M+H]=175.

### PREPARATIVE EXAMPLE 2

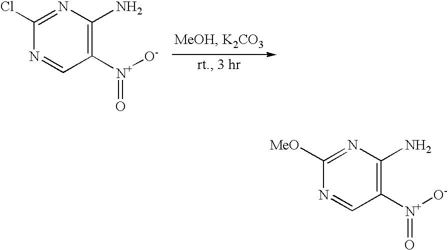

To a solution of 3 g 2-chloro-4-amino-5-nitro-pyrimidine in 100 ml methanol was added 5 g potassium carbonate. The reaction mixture was stirred at rt for 4 hours. The solid was filtered. The filtrate was concentrated to give desired product.

1H NMR (CD₃OD): 9.04 (1H, s); 4.00 (3H, s). LC-MS [M+H]=171.

### PREPARATIVE EXAMPLE 3

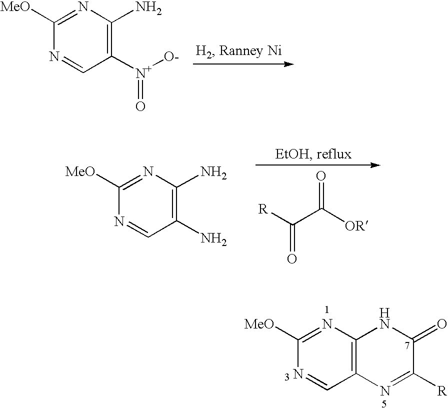

To a solution of 1 g nitropyrimidine in 50 ml ethanol was reduced under 40 psi hydrogen for 1 hour, using 100 mg Ranney Ni as catalyst. The catalyst was filtered through a layer of celite to give 4,5-diamino-pyrimidine. LC-MS [M+H]=141; 1H NMR (CD₃OD): 7.485 (1H, s); 3.808 (3H, s).

The filtrate was added 3-6 eq. ketoester or ketoacid. A couple of drops of sulfrate acid was added to adjust pH 5-6. The reaction mixture was refluxed for 4 hr. The reaction mixture was partitioned between water and ethyl acetate. The organic layer was separated and washed with water, brine. The solvent was removed. The residue was purified by column chromotagraph (silica gel, 7:3 hexanes:ethy lacetate) to give 0.3-1.1 g corresponding 6-R-8-H-pteridinone product.

R=isopropyl, LC-MS [M+H]=221; 1H NMR (CDCl₃): 8.91 (1H, s), 4.10 (3H, s), 3.43-3.58 (1H, m), 1.3 (6H, d).

R=t-butyl, LC-MS [M+H]=235; 1H NMR (CDCl₃): 8.89 (1H, s), 4.09 (3H, s), 1.47 (9H, s).

### EXAMPLE 1

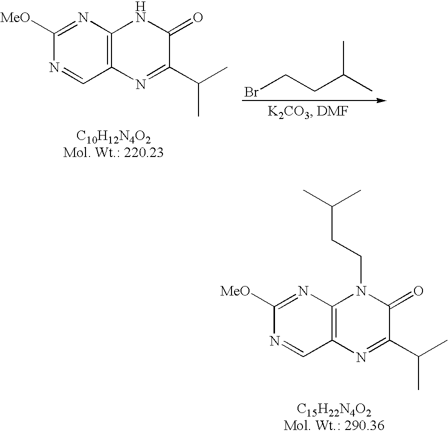

6-isopropyl-8H-pteridinone (50 mg, 0.23 mmol, from preparative example 3) in 1 mL DMF was added 60 mg potasium carbonate and 1.5equiv. of 1-bromo-3-methylbutane. The reaction mixture was stirred at rt for 4 hours. The solid was filtered. The filtrate was concentrated. The residue was purified by column chromotagraphy (silica gel, 4:1 hexanes:ethyl acetate) to give 35 mg desired product.

LC-MS [M+1]=291. 1H NMR (CDCl₃): 8.89 (1H, s), 4.35-4.41 (2H, m), 4.11 (3H, s), 3.53-3.60 (1H, m), 1.66-1.74 (1H, m), 1.63-1.65 (2H, m), 1.31 (6H, d), 1.02 (6H, d).

### EXAMPLE 2

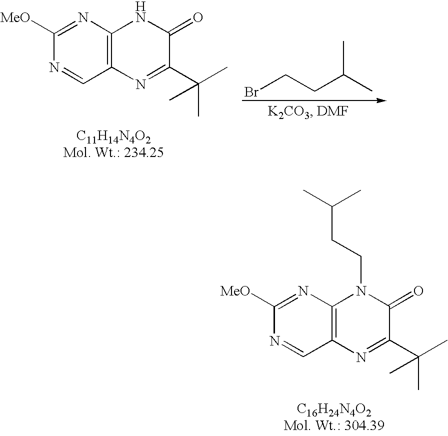

6-t-butyll-8-H-pteridinone (50 mg, 0.21 mmol, from preparative example 3) in 1 mL DMF was added 60 mg potasium carbonate and 1.5equiv. of 1-bromo-3-methylbutane. The reaction mixture was stirred at rt for 4 hours. The solid was filtered. The filtrate was concentrated. The residue was purified by column chromotagraphy (silica gel, 4:1 hexanes:ethyl acetate) to give 30 mg desired product.

LC-MS [M+1]=305.

### EXAMPLE 3

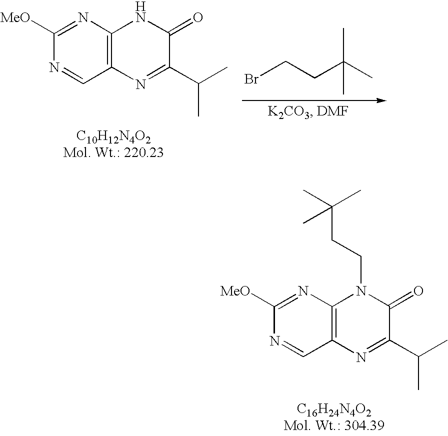

6-isopropyl-8-H-pteridinone (50 mg, 0.23 mmol, from preparative example 3) in 1 mL DMF was added 60 mg potasium carbonate and 1.5equiv. of 1bromo-3,3-dimethylbutane. The reaction mixture was stirred at rt for 4 hours. The solid was filtered The filtrate was concentrated. The residue was purified by column chromotagraphy (silica gel, 4:1 hexanes:ethyl acetate) to give 40 mg desired product.

LC-MS [M+1]=305. 1H NMR (CDCl₃): 8.89 (1H, s), 4.38-4.42 (2H, m), 4.11 (3H, s), 3.53-3.60 (1H, m), 1.61-1.65 (1H, m), 1.31 (6H, d), 1.07 (9H, s).

### EXAMPLE 4

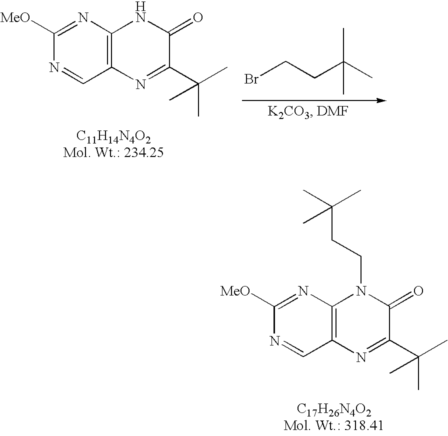

6-t-butyll-8-H-pteridinone (50 mg, 0.21 mmol, from preparative example 3) in 1 mL DMF was added 60 mg potasium carbonate and 1.5equiv. of 1-bromo-3,3-dimethylbutane. The reaction mixture was stirred at rt for 4 hours. The solid was filtered. The filtrate was concentrated. The residue was purified by column chromotagraphy (silica gel, 4:1 hexanes:ethyl acetate) to give 51 mg desired product.

LC-MS [M+1]=319.

Example 5 through 18 as shown below, were made, with some modification, by the alkylation inaccordance with Example 1-4.

### EXAMPLE 5

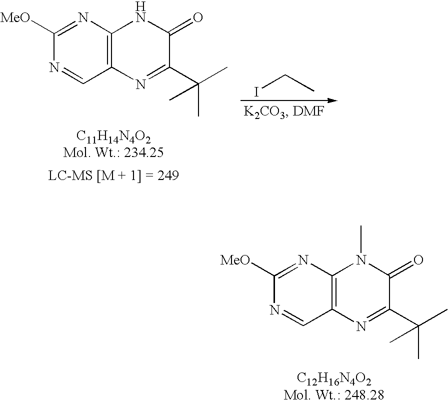

### EXAMPLE 6

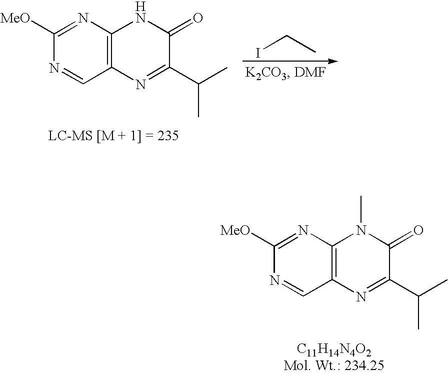

### EXAMPLE 7

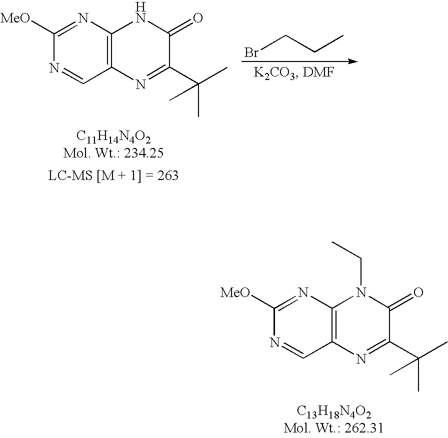

### EXAMPLE 8

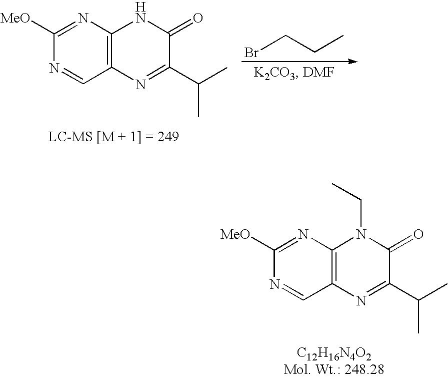

### EXAMPLE 9

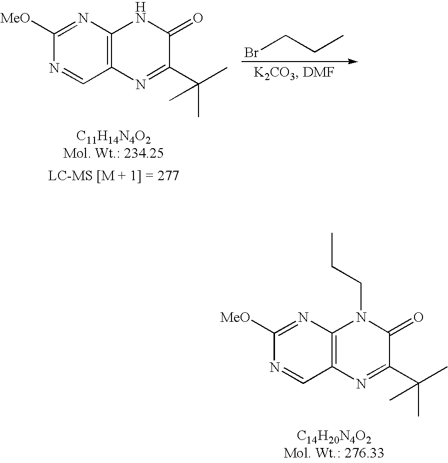

### EXAMPLE 10

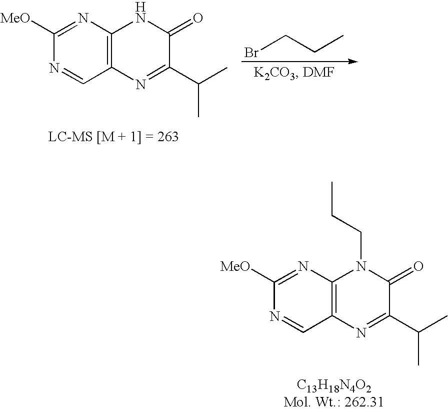

### EXAMPLE 11

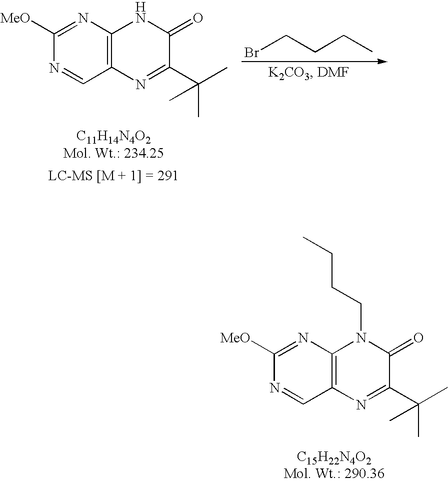

### EXAMPLE 12

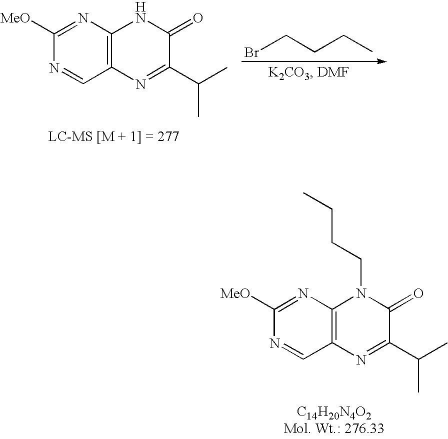

### EXAMPLE 13

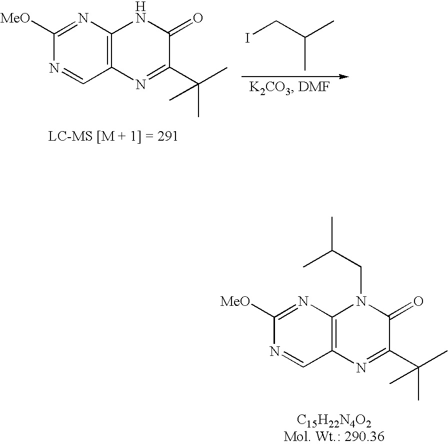

### EXAMPLE 14

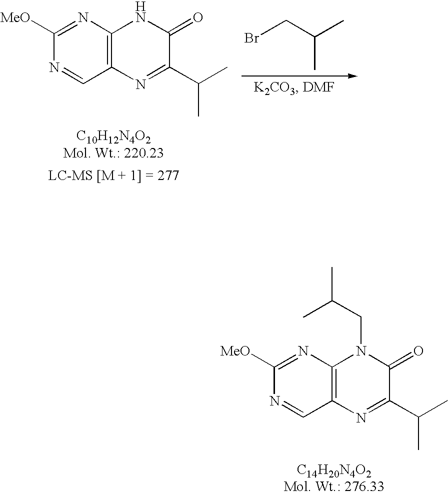

### EXAMPLE 15

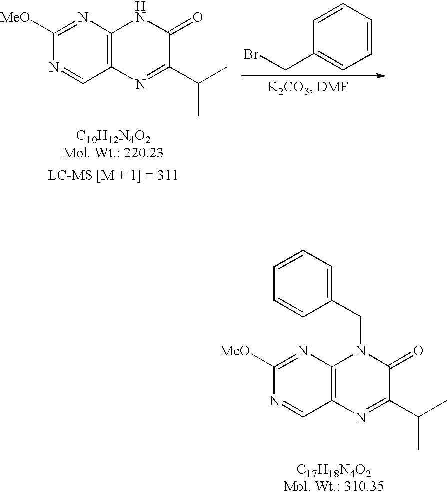

### EXAMPLE 16

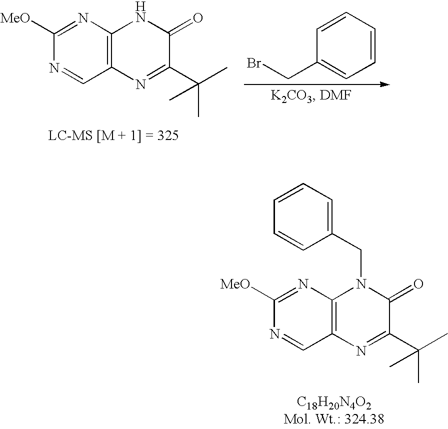

### EXAMPLE 17

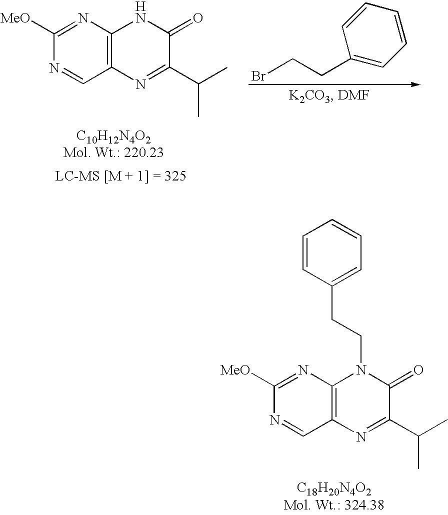

### EXAMPLE 18

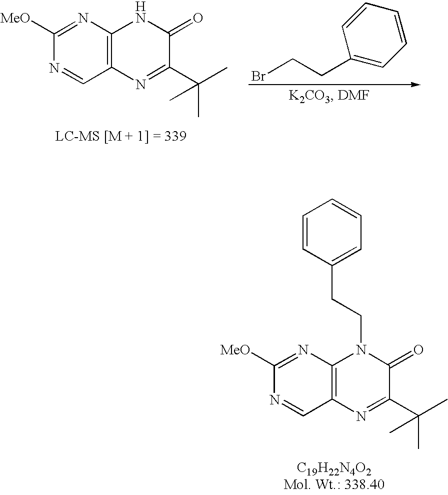

1. A compound of structure formula (I), or a pharmaceutically acceptable salt, enantiomer, diastereomer, in vivo hydrolysable ester or mixture thereof: 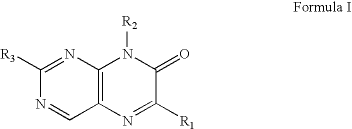
or a pharmaceutically acceptable salt, enantiomer, diastereomer or mixture thereof: wherein,
R₁ represents hydrogen, C₁₋₁₀ alkyl, —C(O)Ra, —(CHRₐ)ₙCONRbRc, —(CH₂)ₙRa, —(CH₂)ₙC₃₋₁₀ heterocyclyl, —(CH₂)ₙC₃₋₈ cycloalkyl, —COORa, aryl, heterocyclyl and alkyl optionally substituted with 1-3 groups selected from R^{a};
R₂ represents hydrogen, C₁₋₁₀ alkyl, C₂₋₆ alkenyl, C₁₋₆ alkylSRa, —(CH₂)ₙO(CH₂)ₘOR, —(CH₂)ₙC₁₋₆ alkoxy, —(CH₂)ₙC₃₋₈ cycloalkyl, —(CH₂)ₙC₃₋₁₀ heterocyclyl, or —(CH₂)ₙRa, said alkyl, heterocyclyl, or aryl optionally substituted with 1-3 groups selected from R^{b};
R₃ represents hydrogen, C₁₋₁₀ alkyl, —(CH₂)ₙC₃₋₈ cycloalkyl, —(CH₂)ₙC₃₋₁₀ heterocyclyl, —(CH₂)ₙNHRₐ, —(CH₂)ₙN(Ra)₂, aryl, C₁₋₆ alkoxy, CF₃, —OCH3, —(CH₂)ₙSO₂Ra, —(CH₂)ₙSO₂N(Ra)₂, —(CH₂)ₙCON(Ra)₂, —(CH₂)ₙCONHC(Ra)₃, nitro, cyano or halogen, said alkyl, alkoxy, heterocyclyl, or aryl optionally substituted with 1-3 groups of R^{a};
Ra represents hydrogen, or C₁₋₁₀ alkyl, C4-12 aryl, C₁₋₁₀ alkyl, —(CH₂)ₙC₃₋₈ cycloalkyl, —(CH₂)ₙC₃₋₁₀ heterocyclyl.
Rb and Rc independently represent H, Ra, C₂₋₆ alkenyl, C₁₋₆ alkylSRa, —(CH₂)ₙO(CH₂)ₘORa, —(CH₂)ₙC₁₋₆ alkoxy, —(CH₂)ₙC₃₋₈ cycloalkyl;
n=1-6

2. A compound of Table 1
TABLE 1

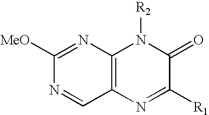

| R2 | R1 |
|---|---|
| Methyl | t-butyl |
| Methyl | Isopropyl |
| Ethyl | t-butyl |
| Ethyl | Isopropyl |
| Propyl | t-butyl |
| Propyl | Isopropyl |
| n-Butyl | Isopropyl |
| n-Butyl | t-butyl |
| Isopropyl | Isopropyl |
| Isopropyl | t-butyl |
| Benzyl | t-butyl |
| Benzyl | Isopropyl |
| 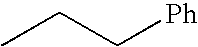 | t-butyl |
| 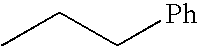 | Isopropyl |
| 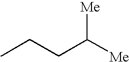 | t-butyl |
| 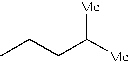 | Isopropyl |
| 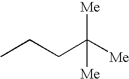 | t-butyl |
| 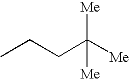 | Isopropyl |

or a pharmaceutically acceptable salt, enantiomer, diastereomer or mixture thereof.

3. A method for treating ocular hypertension or glaucoma comprising administration to a patient in need of such treatment a therapeutically effective amount of a compound of structural formula I: 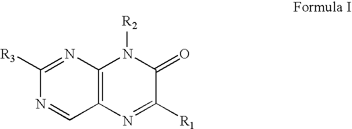
or a pharmaceutically acceptable salt, enantiomer, diastereomer or mixture thereof: wherein,
R₁ represents hydrogen, C₁₋₁₀ alkyl, —C(O)Ra, —(CHRₐ)ₙCONRbRc, —(CH₂)ₙRa, —(CH₂)ₙC₃₋₁₀ heterocyclyl, —(CH₂)ₙC₃₋₈ cycloalkyl, —COORa, aryl, heterocyclyl and alkyl optionally substituted with 1-3 groups selected from R^{a};
R₂ represents hydrogen, C₁₋₁₀ alkyl, C₂₋₆ alkenyl, C₁₋₆ alkylSRa, —(CH₂)ₙO(CH₂)ₘOR, —(CH₂)ₙC₁₋₆ alkoxy, —(CH₂)ₙC₃₋₈ cycloalkyl, —(CH₂)ₙC₃₋₁₀ heterocyclyl, or —(CH₂)ₙRa, said alkyl, heterocyclyl, or aryl optionally substituted with 1-3 groups selected from R^{b};
R₃ represents hydrogen, C₁₋₁₀ alkyl, —(CH₂)ₙC₃₋₈ cycloalkyl, —(CH₂)ₙC₃₋₁₀ heterocyclyl, —(CH₂)ₙNHRₐ, —(CH₂)ₙN(Ra)₂, aryl, C₁₋₆ alkoxy, CF₃, —OCH₃, —(CH₂)ₙSO₂Ra, —(CH₂)ₙSO₂N(Ra)₂, —(CH₂)ₙCON(Ra)₂, —(CH₂)ₙCONHC(Ra)₃, nitro, cyano or halogen, said alkyl, alkoxy, heterocyclyl, or aryl optionally substituted with 1-3 groups of R^{a};
Ra represents hydrogen, or C₁₋₁₀ alkyl, C4-12 aryl, C₁₋₁₀ alkyl, —(CH₂)ₙC₃₋₈ cycloalkyl, —(CH₂)ₙC₃₋₁₀ heterocyclyl.
Rb and Rc independently represent H, Ra, C₂₋₆ alkenyl, C₁₋₆ alkylSRa, —(CH₂)ₙO(CH₂)ₘORa, —(CH₂)ₙC₁₋₆ alkoxy, —(CH₂)ₙC₃₋₈ cycloalkyl;
n=1-6

4. A method according to claim 3 wherein the compound of Formula I is selected from Table:
TABLE 1

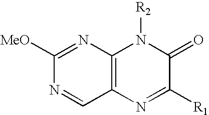

| R2 | R1 |
|---|---|
| Methyl | t-butyl |
| Methyl | Isopropyl |
| Ethyl | t-butyl |
| Ethyl | Isopropyl |
| Propyl | t-butyl |
| Propyl | Isopropyl |
| n-Butyl | Isopropyl |
| n-Butyl | t-butyl |
| Isopropyl | Isopropyl |
| Isopropyl | t-butyl |
| Benzyl | t-butyl |
| Benzyl | Isopropyl |
| 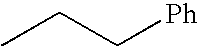 | t-butyl |
| 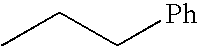 | Isopropyl |
| 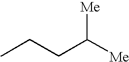 | t-butyl |
| 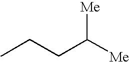 | Isopropyl |
| 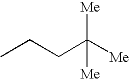 | t-butyl |
| 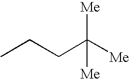 | Isopropyl |

or a pharmaceutically acceptable salt, enantiomer, diastereomer or mixture thereof.

5. A method according to claim 3 wherein the compound of the formula I is administered in a formulation selected from solution topical formulation and a suspension topical formulation.

6. A method for providing a neuroprotective effect comprising administration to a patient in need of such treatment a therapeutically effective amount of a compound of claim 1, or a pharmaceutically acceptable salt, enantiomer, diastereomer or mixture thereof.

7. A method for providing neuroprotective effect to the eye of a mammal in need of such treatment which comprises the step of administering to the mammal a therapeutically effective amount of a pharmaceutical composition which comprises as its active ingredient one or more compounds having maxi-K channel blocking activity.

8. The method of claim 7 wherein the compound having maxi-K channel blocking activity is selected from the structure in formula 1.
